(19) United States
(12) Patent Application Publication
(10) Pub. No.: US 2018/0210925 A1
(43) Pub. Date: Jul. 26, 2018

(54) RELIABILITY MEASUREMENT IN DATA ANALYSIS OF ALTERED DATA SETS

(71) Applicant: KONINKLIJKE PHILIPS N.V., EINDHOVEN (NL)

(72) Inventors: Ushanandini RAGHAVAN, Lexington, MA (US); Daniel Robert ELGORT, New York, NY (US)

(21) Appl. No.: 15/747,784

(22) PCT Filed: Jul. 18, 2016
(86) PCT No.: PCT/IB2016/054255

§ 371 Date: Jan. 26, 2018

Related U.S. Application Data
(60) Provisional application No. 62/198,245, filed on Jul. 29, 2015.

(51) Int. Cl. G06F 17/30; G06F 17/18; G06K 9/62; G16H 50/70

(52) U.S. Cl. CPC G06F 17/30536; G06F 17/30371; G16H 50/70; G06K 9/6201; G06F 17/18

(57) ABSTRACT

Data analysis of altered data includes analyzing (64) a test data set (14) with a data analysis technique using one or more configured processors (30) which create one or more analytical measures, and the test data set selected from an altered data set (12) according to a confidence score. At least one reliability measure of the one or more analytical measure is calculated using the configured one or more processors based on similarity of the one or more analytical measures and same analytic measures created from the data analysis technique applied to one or more reliability test data sets (16, 18) selected from the altered data set according to different confidence scores.

### FIELD OF THE INVENTION

The following generally relates to data analysis and data mining with specific application to data analysis of data sets altered by data cleaning and data integration of healthcare data.

### BACKGROUND OF THE INVENTION

Data mining has been performed on large data sets with data accumulated from a variety of sources. Data mining can include collecting the data, structuring the data, cleaning the data, e.g. removing inconsistencies, correcting errors, integrating or compiling the data from different sources, and analyzing the data for new information. Data from healthcare providers can provide information about patient risk, healthcare treatments, or trends. Data analysis, such as cluster analysis, analysis of variance, and other statistical techniques typically accept the data values as accurate and focus on categorization/classification/prediction with identification and removal of outliers.

As data is modified in preparation for analysis, changes to the data can add uncertainty to the data, which can carry forward to the analysis of the uncertain data. For example, drug names can be misspelled, trade names used, abbreviations used, etc. One approach is to flag any changed data during data cleaning. Reliability of a subsequent analysis is judged based on a percentage of records in an identified group modified by data cleaning, e.g. a high percentage of modified data in an identified cluster from a cluster analysis indicates the cluster is suspect. However, using flags does not discriminate between types of changes to data, some of which are obvious, such as minor misspellings, and some which are less obvious, abbreviations, or alternate names. The process of cleaning the data can introduce new patterns into the cleaned data, which are considered to be spurious, e.g. indicative of the cleaning process, and not reflective of the original data or underlying data patterns.

Another area where uncertainty can be introduced into data, which is subsequently analyzed, is the integration of data from different sources. Healthcare providers are regulated to provide de-identified patient data, i.e. patient identification removed from the data. Sources of data can include different areas from within a healthcare provider, such as patient care records, billing, admission, pharmacy, radiology, etc. Sources can be between different healthcare providers, such as different sites, different hospitals, different outpatient clinics, etc. As data is integrated from the different sources to identify patterns, matching algorithms can add uncertainty, which is carried through to subsequent analysis. For example, de-identified patient diagnoses can be integrated with de-identified pharmacy records. An analysis of drugs prescribed according to diagnosis can include error according to how the patient diagnoses are matched to pharmacy records, e.g. spurious, rather than how patients are prescribed medication based on diagnoses, e.g. not spurious. However, data analysis techniques do not include reliability measures for the data integration, typically only confidence scores or accuracy measures for an applied data analysis technique, such as an R² value in regression analysis/analysis of variance.

### SUMMARY OF THE INVENTION

Aspects described herein address the above-referenced problems and others. The following describes a method and system which determines a reliability measure of an analysis of altered data. The altered data includes confidence scores associated with the data. The confidence scores can be associated with specific instances of data elements altered through data cleaning and/or record instances integrated through data integration.

In one aspect, analysis technique using one or more processors configured which creates one or more analytical measures, and the test data set selected from an altered data set according to a confidence score. At least one reliability measure of the one or more analytical measures is calculated using the configured one or more processors based on similarity of the one or more analytical measures and same analytic measures created from the data analysis technique applied to one or more reliability test data sets selected from the altered data set according to different confidence scores.

In another aspect, a system for data analysis of altered data includes an analysis unit and a reliability unit. The analysis unit includes one or more configured processors which analyze a test data set selected from an altered data set according to a confidence score with a data analysis technique that creates one or more analytical measures, and same analytic measures from the data analysis technique applied to one or more reliability test data sets selected from the altered data set according to different confidence scores. The reliability unit includes the one or more configured processors, which calculate at least one reliability measure of the one or more analytical measures based similarity of the one or more analytical measures and the same analytic measures applied to the one or more reliability test data sets.

In another aspect, a method of data analysis of altered data includes selecting a test data set from an altered data set with a first confidence score greater than a threshold amount, a first reliability test data set with a second confidence score a negative difference from the first confidence score, and a second reliability test set with a third confidence score a positive differences from the first confidence score. The test data set, the first reliability test data set and the second reliability test data set are analyzed with a data analysis technique applied using one or more processors, which create a set of analytical measures, at least one analytical measure for each data set analyzed. A first reliability measure of the at least one analytical measure is calculated based on the at least one analytical measure from the analyzed test data set and the at least one analytical measure from the analyzed first reliability test data set, and a second reliability measure of the at least one analytical measure based on the at least one analytical measure from the analyzed test data set and the at least one analytical measure from the analyzed second reliability test data set.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of reliability measurement in data analysis of altered data sets system.

FIG. 2 illustrates an exemplary report with reliability measurement of a data analysis.

FIG. 3 flowcharts an embodiment of reliability measurement in data analysis of altered data sets.

### DETAILED DESCRIPTION OF EMBODIMENTS

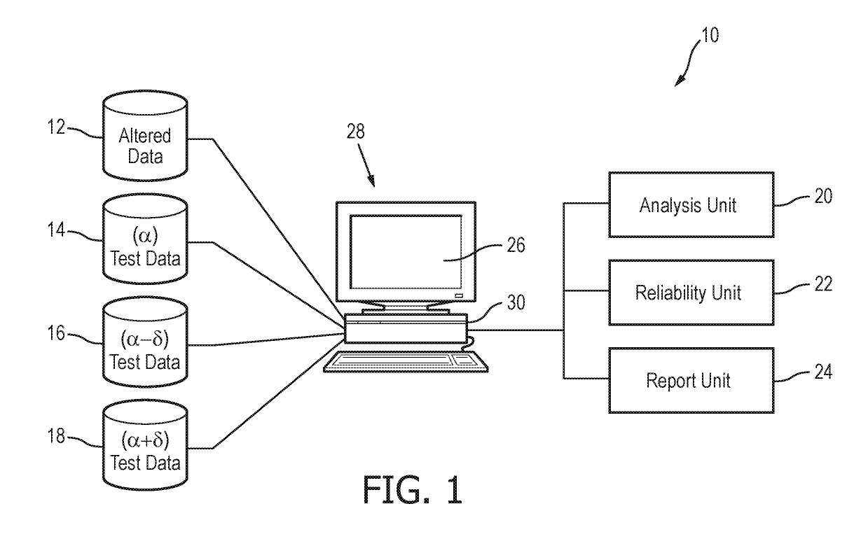

Initially referring to FIG. 1, an embodiment of reliability measurement in data analysis of altered data sets system 10 is schematically illustrated. The system 10 includes an altered data set 12 or electronic access to the altered data set 12 from which a test data set 14 and one or more reliability test data sets 16, 18 are derived. The altered data set 12 includes one or more data elements and/or records which include an associated confidence score. The associated confidence scores can be associated through data cleaning and/or data integration. The confidence scores can be expressed as a continuous range of values, e.g. 0.1-100.0, 0.01-1.00, 1-100, and the like.

For example, occurrences of prescribed drug name: Propofal, Diprivan, Fospropofol, and Propofol are determined to be the same drug name of Propofol in a data set. The name of the drug is a data element or attribute of the prescribed drug. Through data cleaning, different occurrences of the drug names are changed to Propofol and associated with the following confidence scores: (Propofal to Propofol) 98%, (Diprivan to Propofol) 99%, (Fospropofol to Propofol) 25%, and 100% (unchanged). Occurrences of “Propofol” in the data element “drug name” in the altered data set include the associated confidence scores indicative of a confidence that the name change represents the true information. The associated confidence scores can be stored at a record level, e.g. appended to an instance or occurrence, or stored separately, such as a linked or related table. A record includes a group of related data elements, e.g. attributes of a patient. An example technique is more fully described in the Patent Application entitled “System and Method for Uniformly Correlating Unstructured Entry Features to Associated Therapy Features” filed on Dec. 9, 2014, Ser. No. 62/089,336, hereby incorporated by reference in entirety.

Confidence scores associated through data integration are associated at a record level. For example, a first source of data including the following data elements and values: age=63, gender=f, race=Asian, diagnosis=AMI, HR=30, is matched with a second source of data including the following data elements and values: age=64, gender=f, race=Asian, diagnosis=AMI, total chgs=$12,340, outcome=30-day readmission. The match is associated with a confidence score of 73% indicative of the confidence that the match is valid, e.g. that the match is the same patient. The occurrence of the patient identified by the combined data elements of age, gender, race, diagnosis, HR, total chgs, and outcome with the values above is associated with the confidence score of 73%. Other matches or occurrences can be different values. An example technique is more fully described in the Patent Application entitled “Efficient Integration of De-Identified Records” filed on Feb. 27, 2015, Ser. No. 62/121,608, hereby incorporated in entirety.

The test data set 14 include at least one data element with occurrences selected from the altered data set 12 based on one of the confidence measures. For example, selecting occurrences with confidence score associated with “drug name” greater than 75%. The test data set 14 can include a subset of the data elements from the altered data set. For example, the test data set includes age, gender, diagnosis, HR, and outcomes for integration confidence scores is 80% or greater, i.e. a≥80%, where “a” is the confident score for a record occurrence. “total chgs” data element is not included. In another example, the test data set includes age, gender, drug name, and diagnosis where confidence measure of drug name is 75% or more, e.g. a≥75%. The reliability test data sets 16, 18 include the same data elements based on the data analysis and with varied confidence levels, such as α+δ. The test data set 14 and reliability test data sets 16, 18 can be extracted or created from the altered data set 12 using data manipulation techniques known in the art. In one embodiment, the system 10 generates the test data set 14 based on selected data elements and a user modifiable default confidence level, and generates the reliability test data sets 16, 18 with user modifiable default differences in confidence levels. In one embodiment, the data analysis unit 20 performs the data set creation or extraction.

A data analysis unit 20 or a user applies a data analysis using known data analysis techniques, such as descriptive and/or summary statistics, association analysis, clustering analysis, classification, prediction analysis, and the like. The data analysis technique is applied to the test data set 14. For example, a clustering analysis is applied by the data analysis unit to a test data set of age, weight (kg), Heart rate (HR in beats per minute), and creatinine selected with a confidence score greater than 80%, e.g. data integration associated confidence score>a. The same data analysis is applied to each of the reliability test data sets 16, 18 In one embodiment, the reliability test data set 16, 18 generation and analysis is performed automatically with the test data set 12 analysis. In another embodiment, the reliability test data set 16, 18 generation and analysis is performed subsequent to the analysis of the test data set 14 based on a user prompt or user input to perform reliability testing.

A reliability unit 22 computes a reliability measure based on the data analysis of the test data set 12 and the reliability test data sets 16, 17, such as a Jaccard Index for clustering analysis, t-test for descriptive statistics, R² values for predictive analysis, and the like. For example, let clusters C₁, C₂ and C₃ be the result of applying k-means clustering algorithm on the test data set 12, clusters C₁₁, C₁₂, C₁₃ the result of applying the k-means clustering algorithm on the first reliability test data set 16 (X1), and let clusters C₂₁, C₂₂, C₂₃ the result of applying the k-means clustering algorithm on the second reliability test data set 18 (X2). A Jaccard index is calculated for a comparison of {C₁₁, C₁₂, C₁₃} with the original clusters {C₁, C₂, C₃}/_{X1} restricted to records of X₁. If r stands for pairs of data points in the same cluster in both sets, s stands for pairs of data points in the same cluster in X but in different clusters in X₁, and t stands for pairs of data points in the same cluster in X₁ but in different clusters in X, then a Jaccard Index is defined as (r/(r+s+t)). If the index is 1 then the two sets of clusters are identical and when the index is 0 they are completely dissimilar. Values close to 1 can indicate strong similarity between the two solutions. The Jaccard index is calculated for the second test data set 18 (X2). The reliability measure, such as the Jaccard index, can include a range of values, such as 0-100, or the reliability measure can be categorized according to the computed measure.

In another example, such as descriptive statistics, means and/or standard deviations are compared between the test data set 12 and the reliability data sets 16, 18, using a student t-test, or a Welch's t-test. For example, a t-test computes a likelihood that two means are of the same true mean. If a null hypothesis is that the two means are of a different mean, and is not rejected for a t-test comparison of the means of the test data set and the first reliability test data set, and is also not rejected for a t-test comparison of the means for the test data set and the second reliability test data set, then the result is to categorize the composite reliability measure as spurious. If a null hypothesis is not rejected for a t-test of the test data set and the first reliability test data set, and is rejected for a t-test of the test data set and the second reliability test data set, then the result is to categorized as maybe spurious. If the null hypothesis is rejected for both comparisons, then the result is categorized as reliable.

Distributions of data sets can be compared using a Kolmogorov-Smirnov test, e.g. a likelihood that the distributions of each data set represent the same distribution. Predictive models can be compared using accuracy measures, such as R² values. For example, with the same predictors or independent variables, a comparison of R² provides an indication of the a similarity of model fit.

The reliability unit 22 can combine or categorize the reliability measures into a composite measure. In one embodiment the reliability measures can be categorized into or interpreted as categorical measures, such as “reliable”, “may be spurious”, “definitely spurious”. For example, a Jaccard index on a scale of 0.0-1.0 can be categorized as 0.0-0.39, spurious, 0.4-0.69, may be spurious, and 0.7-1.0, reliable. For example using a predictive measure, a relative difference: (R²(X)−R²(X₁))/(R²(X)) change of more than 50% can be categorized as spurious, between 5% and 50%, maybe spurious, and less than 5%, reliable. The categorization ranges and confidence scores can be set according to user preferences, system defaults and/or project preferences, and the like.

A report unit 24 displays the results of the data analysis and the reliability measures. For example, the display can be printed or displayed on a display device 26, such as a display of a computer device 28. The display can include the raw reliability measures, composite measure, and/or categorical measures.

The analysis unit 20, the reliability unit 22, and the report unit 24 comprise at least one processor 30 (e.g., a microprocessor, a central processing unit, digital processor, and the like) configured to executes at least one computer readable instruction stored in a computer readable storage medium, which excludes transitory medium and includes physical memory and/or other non-transitory medium. The processor 30 may also execute one or more computer readable instructions carried by a carrier wave, a signal or other transitory medium. The processor 30 can include local memory and/or distributed memory. The processor 30 can include hardware/software for wired and/or wireless communications. The processor 30 can comprise a computing device 28, such as a desktop computer, a server, a laptop, a mobile device, distributed devices, combinations and the like.

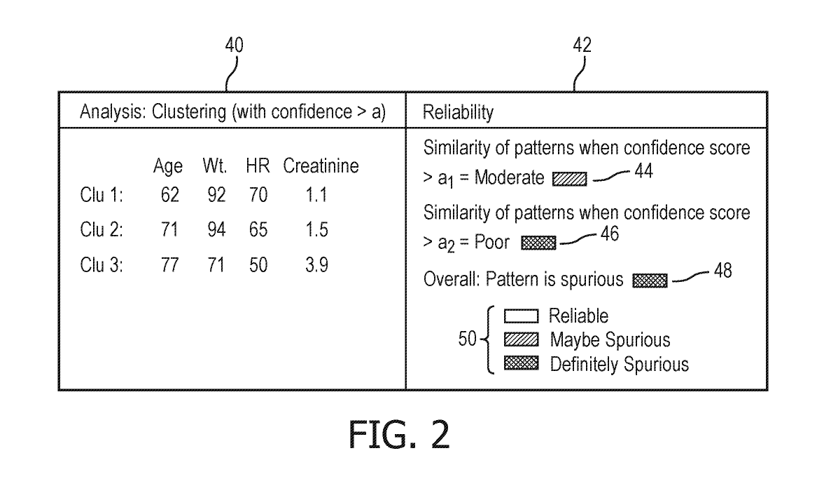

With reference to FIG. 2, an exemplary report with reliability measurement of a data analysis is illustrated. The example report includes a report of the data analysis 40, which is a cluster analysis of a test data set 14 selected with a confidence level (>a) from an altered data set 12. The cluster analysis indicates three identified clusters with data elements or attributes of age in years, weight in kilograms (kg), heart rate in beats per minute (bpm), and creatinine in milligrams/deciliter (mg/dl). A first cluster includes values of 62, 92, 70, and 1.1 for age, weight, heart rate, and creatinine, respectively. A second cluster includes values of 71, 94, 65, and 1.5 respectively, and a third cluster includes values of 77, 71, 50, and 3.9 respectively.

The example report includes a reliability measure 44 of a similarity of the test data set 14 and the first reliability test data set 16, which is presented categorized as moderate or maybe spurious. A second reliability measure 46 is indicative of the similarity between the test data set 14 and the second reliability test data set 18, which is categorized as poor or definitely spurious. A composite measure 48 is shown, which is definitely spurious. A legend 50 indicates the different categories of reliable, maybe spurious, and definitely spurious.

Thus, from the example report with the reliability measures 44, 46, 48, a user can reasonably conclude that the three clusters formed are likely due to patterns introduced as a consequence of data cleaning and/or data integration rather than representing true underlying patterns of the data.

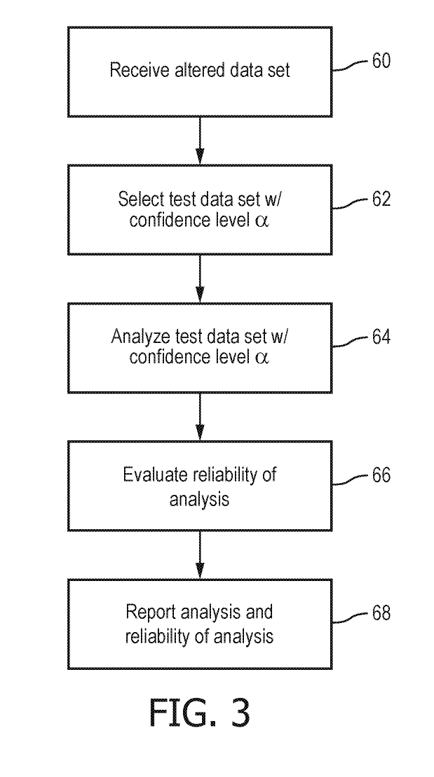

With reference to FIG. 3, an embodiment of reliability measurement in data analysis of an altered data set 12 is flowcharted. At 60 an altered data set 12 is received which includes confidence scores for at least one data element or a set of records. The altered data set 12 can be received by reference, e.g. identification of a location in computer memory and/or storage, or by electronic transmission, e.g. transmitted by network connection from one storage location to another. In one embodiment, the receiving can include cleaning the data and assigning confidences scores to the cleaned/altered data. In one embodiment, the receiving can include integrating two or more sources of data and assigning confidence scores to the integrated data, e.g. records matched or combined. In another embodiment, the receiving can include combinations of data cleaning and data integration.

The test data set 14 is generated at 62 by selecting data from the altered data set 12 with a confidence score above a predetermined threshold. For example, a group of data elements including drug name is selected where a confidence score associated with drug name is more than 70%, e.g. α>70%. In another example, a group of data elements are selected from the altered data set where a confidence score associated with the integrated record is more than 75%.

At 64 the test data set 14 with a confidence score above a predetermined amount (a) is analyzed by the analysis unit 20 using a data analysis technique. The data analysis output at least one analytical measure of the test data set 14, such as clusters, a mean, a standard deviation, an R² value, a class, and the like.

At 66 reliability measures are calculated which evaluate the reliability of the analysis of the test data. The reliability measures are calculated from output analytical measures of the same analysis of the first reliability data set 16 selected with the same data elements as the test data set 12 and a confidence score with a negative difference from the predetermined score (α−δ), and output analytical measures of the same analysis of the second reliability data set 18 with a confidence score a positive difference from the predetermined score (α−δ). The reliability measure includes raw measures of the similarity of the output analytical measures, such as the Jaccard Index, T-test, and the like. The reliability measure can be categorized and/or combined into a composite measure. In one embodiment, the analytical measures of the reliability data sets 16, 18 and the reliability measures are calculated in response to a significant output analytical measure from the analysis of the test data set 14. In another embodiment, the analytical measures are calculated in parallel to the analysis of the test data set 14, and the reliability measures calculate subsequent to the output of the analytical measures.

At 68 the reliability measures are reported. The reliability measures can be reported as raw measures, categorized raw measures, composite measures, or categorized composite measures. The reporting can be presented with the output analytical measures of the test data set 14 on the display device or incorporated in an electronic or printed file for subsequent review.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method of data analysis of altered data, comprising:
analyzing a test data set with a data analysis technique using configured one or more processors which creates one or more analytical measures, and the test data set selected from an altered data set according to a confidence score;
calculating at least one reliability measure of the one or more analytical measures using the configured one or more processors based on a similarity of the one or more analytical measures and same analytic measures created from the data analysis technique applied to one or more reliability test data sets selected from the altered data set according to different confidence scores.

2. The method according to claim 1, wherein the reliability measures include at least one of a Jaccard Index, a student t-test, a Welch's t-test, a Kolmogorov-Smirnov test, or a predictive model accuracy measure.

3. The method according to claim 1, further including:
altering data within the altered data set for at least one data element by changing values in the altered data set and associating the confidence score with the changed values.

4. The method according to claim 1 further including:
integrating data into the altered data set by matching records from at least two sources and associating the confidence score with the integrated data.

5. The method according to claim 1, wherein the analytical measure includes at least one of a descriptive statistic, a predictive accuracy measure, a classification, or a data distribution.

6. The method according to claim 1, wherein the calculating at least one reliability measure includes:
calculating a first reliability measure based on the data analysis of a first reliability test data set selected from the altered data set with a first confidence score which is different from the confidence score; and
calculating a second reliability measure based on the data analysis of a second reliability test data set selected from the altered data with a second confidence score which is different from the confidence score and the first confidence score.

7. The method according to claim 6, wherein the first confidence score is a negative difference from the confidence score, and the second confidence score is a positive difference from the confidence score.

8. The method according to claim 1, wherein the at least one reliability measure includes a composite measure which is a function of individual reliability measures.

9. The method according to claim 1, wherein the at least one reliability measure is further categorized.

10. The method according to claim 1, wherein analyzing the test data set with the data analysis technique includes:
applying the data analysis technique in parallel to the test data set and the one or more reliability test data sets.

11. The method according to claim 1, further including:
outputting the reliability analysis to one of a display device, a printing device, or a computer file.

12. A system for data analysis of altered data, comprising:
an analysis unit comprising one or more configured processors which analyzes a test data set selected from an altered data set according to a confidence score with a data analysis technique that creates one or more analytical measures, and same analytic measures from the data analysis technique applied to one or more reliability test data sets selected from the altered data set according to different confidence scores;
a reliability unit comprising the one or more configured processors, which calculates at least one reliability measure of the one or more analytical measures based similarity of the one or more analytical measures and the same analytic measures applied to the one or more reliability test data sets.

13. The system according to claim 12, wherein the reliability measures include at least one of a Jaccard Index, a student t-test, a Welch's t-test, a Kolmogorov-Smirnov test, or a predictive model accuracy measure.

14. The system according to claim 12, wherein the confidence score is associated with the altered data set according to changed data values.

15. The system according to claim 12, wherein the confidence score is associated with the altered data according to data integrated into the altered data set by matching records from at least two sources.

16. The system according to claim 12, wherein the analytical measure includes at least one of a descriptive statistic, a predictive accuracy measure, a classification, or a data distribution.

17. The system according to claim 12, wherein the reliability unit calculates a first reliability measure based on the data analysis of a first reliability test data set selected from the altered data set with a first confidence score which is different from the confidence score, and calculates a second reliability measure based on the data analysis of a second reliability test data set selected from the altered data with a second confidence score which is different from the confidence score and the first confidence score.

18. The system according to claim 12, wherein the reliability unit categorizes the at least one reliability measure.

19. The system according to claim 12, wherein the analysis applies the data analysis technique in parallel to the test data set and the one or more reliability test data sets.

20. A method of data analysis of altered data, comprising:
selecting a test data set from an altered data set with a first confidence score greater than a threshold amount, a first reliability test data set with a second confidence score a negative difference from the first confidence score, and a second reliability test set with a third confidence score a positive differences from the first confidence score;
analyzing the test data set, the first reliability test data set and the second reliability test data set with a data analysis technique applied using one or more processors which creates a set of analytical measures, at least one analytical measure for each data set analyzed;
calculating a first reliability measure of the at least one analytical measure based on the at least one analytical measure from the analyzed test data set and the at least one analytical measure from the analyzed first reliability test data set, and a second reliability measure of the at least one analytical measure based on the at least one analytical measure from the analyzed test data set and the at least one analytical measure from the analyzed second reliability test data set.
